(19) United States
(12) Patent Application Publication
(10) Pub. No.: US 2006/0041237 A1
(43) Pub. Date: Feb. 23, 2006

(54) Method and apparatus for displaying support for an entity

(76) Inventors: Scott C. Raffone, Northford, CT (US); Mark L. Turcio, Durham, CT (US)

Correspondence Address: O'SHEA, GETZ & KOSAKOWSKI, P.C., 1500 MAIN ST., SUITE 912, SPRINGFIELD, MA, 01115, US

(21) Appl. No.: 10/966,918

(22) Filed: Oct. 15, 2004

Related U.S. Application Data
(60) Provisional application No. 60/602,379, filed on Aug. 18, 2004.

(51) Int. Cl. A61F 13/15

(52) U.S. Cl. 604/361

(57) ABSTRACT

A method and apparatus for displaying support for an entity is provided. The apparatus is an absorbent article that includes an outer cover, an absorbent assembly, a first graphic, and a second graphic. The outer cover has an exterior surface and an interior surface, and both surfaces extend between a front portion and a rear portion. The absorbent assembly has a first surface and a second surface, and the first surface is disposed adjacent the interior surface. The first graphic is disposed on the exterior surface and is associated with a first entity. The second graphic is disposed on the second surface of the absorbent assembly, and the second graphic is associated with a second entity. The first entity and the second entity are related organizations. The method includes the steps of: (a) providing an absorbent article that includes an outer cover with an exterior surface and an interior surface, an absorbent assembly with a first surface and a second surface, wherein the first surface is disposed adjacent the interior surface; (b) displaying a first graphic on the exterior surface, which first graphic is associated with a first entity; and (c) displaying a second graphic disposed on the second surface of the absorbent assembly, which second graphic is associated with a second entity, wherein the first entity and the second entity are related organizations.

### CROSS REFERENCE TO RELATED APPLICATIONS

This application claims the benefits of prior filed, co-pending provisional U.S. patent application Ser. No. 60/602379 filed Aug. 18, 2004.

### BACKGROUND OF THE INVENTION

1. Field of the Invention

The present invention relates to methods and apparatus for displaying support for an entity in general, and to methods and apparatus for displaying information on an absorbent article of clothing in particular.

2. Background Information

Some disposable absorbent articles, such as diapers and adult incontinence products, are marketed today with an image or pattern visible on the outer surface of the article. The outer surface of the interior of the article (i.e., the surface that faces the body of the wearer) is typically without visible images or patterns. It is desirable to provide a method and an apparatus for displaying support for an entity on a piece of clothing.

### DISCLOSURE OF THE INVENTION

According to an aspect of the present invention, an absorbent article is provided that includes an outer cover, an absorbent assembly, a first graphic, and a second graphic. The outer cover has an exterior surface and an interior surface, and both surfaces extend between a front portion and a rear portion. The absorbent assembly has a first surface and a second surface, and the first surface is disposed adjacent the interior surface. The first graphic is disposed on the exterior surface and is associated with a first entity. The second graphic is disposed on the second surface of the absorbent assembly, and the second graphic is associated with a second entity. The first entity and the second entity are related organizations.

According to another aspect of the present invention, a method for displaying support for an entity is provided that comprises the steps of: (a) providing an absorbent article that includes an outer cover with an exterior surface and an interior surface, an absorbent assembly with a first surface and a second surface, wherein the first surface is disposed adjacent the interior surface; (b) displaying a first graphic on the exterior surface, which first graphic is associated with a first entity; and (c) displaying a second graphic disposed on the second surface of the absorbent assembly, which second graphic is associated with a second entity, wherein the first entity and the second entity are related organizations.

An advantage of the present invention is that it provides a medium and method for showing different levels of support for two different entities.

Another advantage of the present invention is that it can be used for a variety of different entity types; e.g., sports teams, colleges, political candidates, etc.

These and other objects, features and advantages of the present invention will become apparent in light of the detailed description of the best mode embodiment thereof, as illustrated in the accompanying drawings.

### BRIEF DESCRIPTION OF THE DRAWINGS

FIG. 1 is a diagrammatic view of an absorbent article embodiment, illustrating the rear portion of the exterior of the article.

FIG. 2 is a diagrammatic view of an absorbent article embodiment, illustrating the exposed second surface of the absorbent assembly.

### DETAILED DESCRIPTION OF THE INVENTION

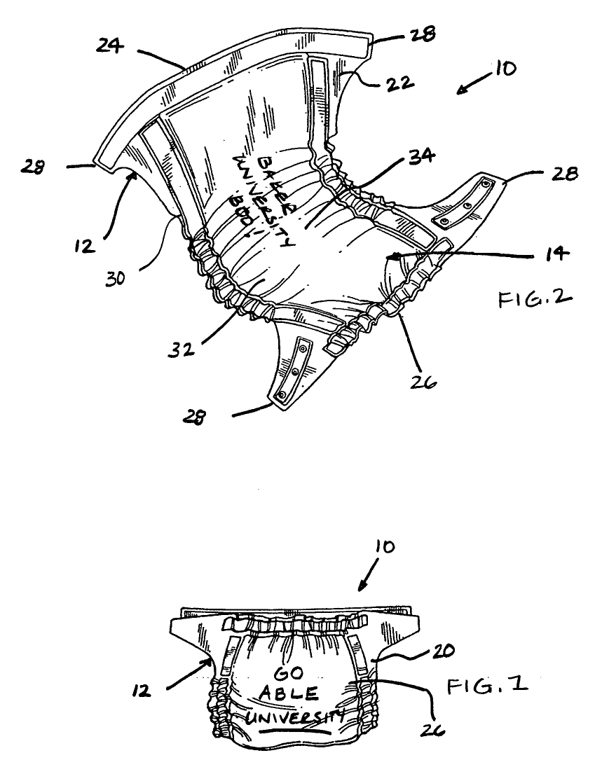

Referring to FIGS. 1-2, a method and apparatus for displaying support for an entity is provided. The apparatus and method include an absorbent article 10 that includes an outer cover 12, an absorbent assembly 14, a first graphic 16, and a second graphic 18.

The outer cover 12 includes an exterior surface 20 and an interior surface 22, which surfaces 20,22 extend between a front portion 24 and a rear portion 26. The outer cover 12 shown in FIG. 2 is hourglass-shaped with tabs 28 for attaching the rear portion 26 and the front portion 24 together. Once connected, apertures for receiving the legs of the user are formed relative to the front portion 24 and rear portion 26. The absorbent assembly 14 has a first surface 30 and a second surface 32. The first surface 30 is disposed adjacent the interior surface 22, and the second surface 32 is exposed. The absorbent assembly 14 is operable to absorb liquids, moisture, etc. There are many known absorbent articles capable as being worn as an article of clothing, and the present application is not limited to any particular embodiment.

The first graphic 16 is displayed on the exterior surface 20 of the outer cover 12, and is associated with a first entity. The position of the first graphic 16 on the exterior surface 20 can vary to suit the application. For example, FIG. 1 shows a first graphic of “University Able” positioned on the rear portion 26 of the absorbent article 10 in a manner such that the text of the first graphic 16 is oriented upright when the absorbent article 10 is worn by the user, and therefore readily understood. The second graphic 18 is displayed on the second surface 32 of the absorbent assembly 14, and is associated with a second entity. For example, FIG. 2 shows a second graphic of “University Baker” disposed in a middle portion 34 of the absorbent article 10 between the front portion 24 and the rear portion 26. The first entity and the second entity are related organizations.

The term “entity” is used herein to refer to a sports team, a business organization, an educational institute (e.g., a high school, university, college, etc.), a political party or other organization having a following of supporters.

The term “graphic” is used herein to refer to a symbol, name, logo, design, or the like that is associated with an entity. For example, the depiction of a sports team name in text, design, or combination thereof, is an example of a graphic. Another example of a graphic is the depiction of the symbol of a political party.

The term “related organization” is used herein to mean that first and second entities have a common interest. For example, the first and second entities may both be a university or a college. As another example, the first and second entities may both be a sports team. In a preferred embodiment, the first and second entities compete against one another, and in a most preferred embodiment the entities are rivals; e.g., University Able and University Baker have rival sports programs. In some embodiments, there may be more than one second entity or first entity. For example, a second graphic 18 displayed on the second surface 32 of the absorbent assembly 14 may identify or be associated with more than one second entity; e.g., several members of a sports conference may be identified within the second graphic 18.

Although this invention has been shown and described with respect to the detailed embodiments thereof, it will be understood by those skilled in the art that various changes in form and detail thereof may be made without departing from the spirit and the scope of the invention.

What is claimed is:

1. An absorbent article, comprising:
an outer cover, having an exterior surface and an interior surface;
an absorbent assembly, having a first surface and a second surface, wherein the first surface is disposed adjacent the interior surface;
a first graphic disposed on the exterior surface associated with a first entity; and
a second graphic disposed on the second surface of the absorbent assembly, the second graphic associated with a second entity;
wherein the first entity and the second entity are related organizations.

2. An absorbent article according to claim 1, wherein the first entity is a first sports team and the second entity is a second sports team.

3. An absorbent article according to claim 2, wherein the first sports team and the second sports team play the same sport.

4. An absorbent article according to claim 1, wherein the first entity is a first educational institute and the second entity is a second educational institute.

5. An absorbent article according to claim 4, wherein the first educational institute and the second educational institute compete against one another.

6. An absorbent article according to claim 1, wherein the exterior surface and the interior surface of the outer cover extend between a front portion and a rear portion, and the first graphic is disposed on the rear portion of the exterior surface of the outer cover.

7. An absorbent article, comprising:
an outer cover, having an exterior surface and an interior surface;
an absorbent assembly, having a first surface and a second surface, wherein the first surface is disposed adjacent the interior surface;
a first graphic disposed on the exterior surface associated with a first entity; and
a second graphic disposed on the second surface of the absorbent assembly, the second graphic associated with a second entity;
wherein the first entity and the second entity are rivals.

8. An absorbent article, comprising:
an outer cover, having an exterior surface and an interior surface;
an absorbent assembly, having a first surface and a second surface, wherein the first surface is disposed adjacent the interior surface;
a first graphic disposed on the exterior surface associated with a first sports team; and
a second graphic disposed on the second surface of the absorbent assembly, the second graphic associated with a second sports team.

9. The absorbent article of claim 8, wherein the first sports team and the second sports team play the same sport.

10. An absorbent article according to claim 1, wherein the first entity is a first political party and the second entity is a second political party.

11. A method for displaying support for an entity, comprising the steps of:
providing an absorbent article that includes an outer cover with an exterior surface and an interior surface, an absorbent assembly with a first surface and a second surface, wherein the first surface is disposed adjacent the interior surface;
displaying a first graphic on the exterior surface, which first graphic is associated with a first entity; and
displaying a second graphic disposed on the second surface of the absorbent assembly, which second graphic is associated with a second entity;
wherein the first entity and the second entity are related organizations.

12. A method according to claim 11, wherein the first entity is a first sports team and the second entity is a second sports team.

13. A method according to claim 12, wherein the first sports team and the second sports team play the same sport.

14. A method according to claim 10, wherein the first entity is a first educational institute and the second entity is a second educational institute.

15. A method according to claim 14, wherein the first educational institute and the second educational institute compete against one another.
